(19) United States
(12) Patent Application Publication
(10) Pub. No.: US 2018/0056210 A1
(43) Pub. Date: Mar. 1, 2018

(54) SEPARATION OF (Z)-1-CHLORO-3,3,3-TRIFLUOROPROPENE (HCFO-1233zd(Z)) AND 1-CHLORO-1,3,3,3-TETRAFLUOROPROPANE (HCFC-244fa) BY ADDING A THIRD COMPONENT

(71) Applicant: Honeywell International Inc., Morris Plains, NJ (US)

(72) Inventors: Konstantin A. Pokrovski, Orchard Park, NY (US); Daniel C. Merkel, Orchard Park, NY (US); Rajat S. Basu, East Amherst, NY (US); Hsueh Sung Tung, Getzville, NY (US)

(21) Appl. No.: 15/688,254

(22) Filed: Aug. 28, 2017

Related U.S. Application Data
(60) Provisional application No. 62/382,492, filed on Sep. 1, 2016.

(51) Int. Cl. B01D 3/36; C07C 19/10; C07C 21/18; C01B 7/19

(52) U.S. Cl. CPC B01D 3/36; C01B 7/191; C07C 21/18; C07C 19/10

(57) ABSTRACT

A method for separating halocarbons and, in particular, a method for separating (Z)-1-chloro-3,3,3-trifluoropropene (HCFO-1233zd(Z), or simply 1233zd(Z)) and 1-chloro-1,3,3,3-tetrafluoropropane (HCFC-244fa, or simply 244fa) via distillation by adding a third component, hydrogen fluoride (HF), forming a binary azeotrope of 1233zd(Z) and HF. The binary 1233zd(Z)/HF azeotrope may then be recovered from the distillation column as an overhead stream which includes only a relatively minor amount of 244fa, while the 244fa may be recovered from the distillation column as a bottoms stream which includes only relatively minor amounts of 1233zd(Z) and HF.

### CROSS REFERENCE TO RELATED APPLICATIONS

This application claims the benefit under Title 35, U.S.C. §119(e) of U.S. Provisional Patent Application Ser. No. 62/382,492, filed on Sep. 1, 2016, entitled “Separation of (Z)-1-chloro-3,3,3-trifluoropropene (HCFO-1233zd(Z)) and 1-chloro-1,3,3,3-tetrafluoropropane (HCFC-244fa) by adding a third component”, the entire disclosure of which is expressly incorporated herein by reference

### FIELD OF THE INVENTION

The present invention provides a method for separating halocarbons. In particular, the present invention provides a method for separating (Z)-1-chloro-3,3,3-trifluoropropene (HCFO-1233zd(Z)) and 1-chloro-1,3,3,3-tetrafluoropropane (HCFC-244fa) via distillation with the addition of a third component.

### DESCRIPTION OF THE PRIOR ART

Fluorocarbon based fluids have found widespread use in industry in a number of applications, including as refrigerants, aerosol propellants, blowing agents, heat transfer media, and gaseous dielectrics. Due to suspected environmental problems associated with the use of some of these fluids, including the relatively high global warming potentials associated therewith, it is desirable to use fluids having the lowest possible global warming potential (GWP) in addition to also having zero ozone depletion potential (ODP). Thus, there is considerable interest in developing environmentally friendlier materials for the applications mentioned above.

Hydrochlorofluoroolefins (HCFOs) having zero ozone depletion and low global warming potential have been identified as potentially filling this need. However, the toxicity, boiling point, and other physical properties of such chemicals vary greatly from isomer to isomer. One HCFO having valuable properties is (Z)-1-chloro-3,3,3-trifluoropropene) (HCFO-1233zd(Z)), which has been proposed as a next generation non ozone depleting and low global warming potential solvent. HCFO-1233zd(Z) is also an important intermediate in the production of (E)-1-chloro-3,3,3-trifluoropropene (HCFO-1233zd(E)) which is also a commercial non ozone depleting and low global warming potential foam blowing agent and solvent.

Thus, 1233zd has two isomers: (E) and (Z). HCFO-1233zd(E) or (E)-1-chloro-3,3,3-trifluoropropene has a boiling point of approximately 19° C. while HCFO-1233zd(Z) or (Z)-1-chloro-3,3,3-trifluoropropene has a boiling point of approximately 39° C. While 1233zd(E) is a HCFO having valuable properties, in some instances, a zero ozone depletion and low global warming potential HCFO with a higher boiling point may be desired. Thus, 1233zd(Z) may be a suitable HCFO when higher boiling points are desired.

The processes for the manufacture of HCFO-1233zd(Z) produces HCFC-244fa as a by-product. HCFC-1233zd(Z) and HCFC-244fa are also intermediates in the production of HCFO-1233zd(E), as described in U.S. Pat. Nos. 7,829,747, 8,217,208, 8,835,700, and 9,045,386, the specifications of which are incorporated herein by reference. HCFO-1233zd(Z) and HCFC-244fa have been disclosed to be effective refrigerants, heat transfer mediums, propellants, foaming agents, blowing agents, gaseous dielectrics, sterilant carriers, polymerization mediums, particulate removal fluids, carrier fluids, buffing abrasive agents, displacement drying agents and power cycle working fluids.

However the boiling points of HCFO-1233zd(Z) and HCFC-244fa are very close to one another, specifically, HCFO-1233zd(Z) has a normal boiling point (NBP) of 39.5° C. and HCFC-244fa has a normal boiling point (NBP) of 42.2° C. Therefore, HCFO-1233zd(Z) and HCFC-244fa are not separable by conventional separation techniques such as distillation.

What is needed is an effective means for separating HCFO-1233zd(Z) and HCFC-244fa from one another.

### SUMMARY OF THE INVENTION

The present invention provides a method for separating halocarbons. In particular, the present invention provides a method for separating (Z)-1-chloro-3,3,3-trifluoropropene (HCFO-1233zd(Z), or simply 1233zd(Z)) and 1-chloro-1,3,3,3-tetrafluoropropane (HCFC-244fa, or simply 244fa) via distillation by adding a third component, hydrogen fluoride (HF), forming a binary azeotrope of 1233zd(Z) and HF. The binary 1233zd(Z)/HF azeotrope may then be recovered from the distillation column as an overhead stream which includes only a relatively minor amount of 244fa, while the 244fa may be recovered from the distillation column as a bottoms stream which includes only relatively minor amounts of 1233zd(Z) and HF.

In one form thereof, the present invention provides a method of separating (Z)-1-chloro-3,3,3-trifluoropropene (HCFO-1233zd(Z)) and 1-chloro-1,3,3,3-tetrafluoropropane (HCFC-244fa), including the steps of: providing a mixture of 1233zd(Z) and 244fa to a distillation column; adding an amount of hydrogen fluoride (HF) to the distillation column to form an azeotropic or azeotrope-like mixture consisting essentially of 1233zd(Z) and HF; distilling the 244fa and the azeotrope-like mixture of 1233zd(Z) and HF; and recovering the azeotropic or azeotrope-like mixture of 1233zd(Z) and HF in an overhead stream.

In the foregoing method, the method may include, after the distilling step, the additional step of recovering 244fa in a bottoms stream. The step of providing a mixture of 1233zd(Z) and 244fa may further include providing a mixture of 1233zd(Z) and 244fa having between 5.0 wt. % and 98.0 wt. % 1233zd(Z) and between 2.0 wt. % and 95.0 wt. % 244fa, based on a combined weight of 1233zd(Z) and 244fa. The step of adding an amount of hydrogen fluoride (HF) may further include adding between 1.0 wt. % and 34.3 wt. % HF, based on a combined weight of 1233zd(Z), 244fa, and HF. The distilling step may be conducted at a pressure between 1 psig and 214.7 psig.

Also, in the foregoing method, the azeotropic or azeotrope-like mixture of 1233zd(Z) and HF may have a boiling point of about 0-60° C. at a pressure of about 3-73 psis. The step of recovering the azeotropic or azeotrope-like mixture of 1233zd(Z) and HF in an overhead stream may further encompass recovering less than 5.0 wt. % 244fa in the overhead stream or less than 1.0 wt. % 244fa in the overhead stream. The step of recovering 244fa in a bottoms stream may further encompass recovering 244fa that includes less than 20.0 wt. % 1233zd(Z) or less than 10.0 wt. % 1233zd(Z). The method may be a continuous process and may further include the additional steps of repeating the recovering steps and adding an additional amount of HF to make up for HF removed in the recovering steps.

Also, in the foregoing method, the method may include, after the step of recovering the azeotropic or azeotrope-like mixture of 1233zd(Z) and HF in an overhead stream, the additional step of separating the 1233zd(Z) and HF. The method may still further include, after the step of recovering 244fa, an additional step selected from the group consisting of: subjecting the recovered 244fa to a dehydrofluorination reaction to convert the 244fa to (E)-1-chloro-3,3,3-trifluoropropene (HCFO-1233zd(E)); and subjecting the recovered 244fa to a fluorination reaction to convert the 244fa to 1,1,1,3,3-pentafluoropropane (HFC-245fa).

In a further form thereof, the present invention provides a method of separating (Z)-1-chloro-3,3,3-trifluoropropene (HCFO-1233zd(Z)) and 1-chloro-1,3,3,3-tetrafluoropropane (HCFC-244fa), including the steps of: providing a mixture of 1233zd(Z) and 244fa to a distillation column, the mixture including between 5.0 wt. % and 98.0 wt. % 1233zd(Z) and between 2.0 wt. % and 95.0 wt. % 244fa, based on a combined weight of 1233zd(Z) and 244fa; adding between 1.0 wt. % and 34.3 wt. % hydrogen fluoride (HF), based on a combined weight of 1233zd(Z), 244fa, and HF, to the distillation column to form an azeotropic or azeotrope-like mixture consisting essentially of 1233zd(Z) and HF having a boiling point of about 0-60° C. at a pressure of about 3-73 psia; distilling the 244fa and the azeotrope-like mixture of 1233zd(Z) and HF; recovering the azeotropic or azeotrope-like mixture of 1233zd(Z) and HF in an overhead stream; and recovering 244fa in a bottoms stream.

In the foregoing method, the step of recovering the azeotropic or azeotrope-like mixture of 1233zd(Z) and HF in an overhead stream may further include recovering less than 5.0 wt. % 244fa in the overhead stream. The step of recovering the azeotropic or azeotrope-like mixture of 1233zd(Z) and HF in an overhead stream may further include recovering less than 1.0 wt. % 244fa in the overhead stream. The step of recovering 244fa in a bottoms stream may include recovering 244fa that includes less than 20.0 wt. % 1233zd(Z). The step of recovering 244fa in a bottoms stream may include recovering 244fa that includes less than 10.0 wt. % 1233zd(Z).

Further, in the foregoing method, the method may include, after the step of recovering the azeotropic or azeotrope-like mixture of 1233zd(Z) and HF in an overhead stream, the additional step of separating the 1233zd(Z) and HF. The method may also include, after the step of recovering 244fa, an additional step selected from the group consisting of: subjecting the recovered 244fa to a dehydrofluorination reaction to convert the 244fa to (E)-1-chloro-3,3,3-trifluoropropene (HCFO-1233zd(E)); and subjecting the recovered 244fa to a fluorination reaction to convert the 244fa to 1,1,1,3,3-pentafluoropropane (HFC-245fa).

It should be appreciated by those persons having ordinary skill in the art(s) to which the present invention relates that any of the features described herein in respect of any particular aspect and/or embodiment of the present invention can be combined with one or more of any of the other features of any other aspects and/or embodiments of the present invention described herein, with modifications as appropriate to ensure compatibility of the combinations. Such combinations are considered to be part of the present invention contemplated by this disclosure.

It is to be understood that both the foregoing general description and the following detailed description are exemplary and explanatory only and are not restrictive of the invention as claimed. Other embodiments will be apparent to those skilled in the art from consideration of the specification and practice of the invention disclosed herein.

### BRIEF DESCRIPTION OF THE DRAWINGS

The above mentioned and other features of the invention, and the manner of attaining them, will become more apparent and the invention itself will be better understood by reference to the following description of embodiments of the invention taken in conjunction with the accompanying drawings.

FIG. 1 is a schematic representation of the present process.

Although the drawings represent embodiments of various features and components according to the present disclosure, the drawings are not necessarily to scale and certain features may be exaggerated in order to better illustrate and explain the present disclosure. The exemplification set out herein illustrates an embodiment of the invention, and such exemplification is not to be construed as limiting the scope of the invention in any manner.

### DETAILED DESCRIPTION

The present invention provides a method for separating halocarbons. In particular, the present invention provides a method for separating (Z)-1-chloro-3,3,3-trifluoropropene (HCFO-1233zd(Z), or simply 1233zd(Z)) and 1-chloro-1,3,3,3-tetrafluoropropane (HCFC-244fa, or simply 244fa) via distillation by adding a third component, hydrogen fluoride (HF), forming a binary azeotrope of 1233zd(Z) and HF. The binary 1233zd(Z)/HF azeotrope may then be recovered from the distillation column as an overhead stream which includes only a relatively minor amount of 244fa, while the 244fa may be recovered from the distillation column as a bottoms stream which includes only relatively minor amounts of 1233zd(Z) and HF.

It has been surprisingly discovered that the formation of a binary azeotrope of 1233zd(Z) and HF changes the relative volatility of the foregoing components to a sufficient extent to allow separation of the 1233zd(Z)/HF azeotrope from 244fa by distillation. Advantageously, via the present method, separation of 1233zd(Z) and 244fa may be achieved using commonly sized and commercially feasible conventional fractional distillation equipment.

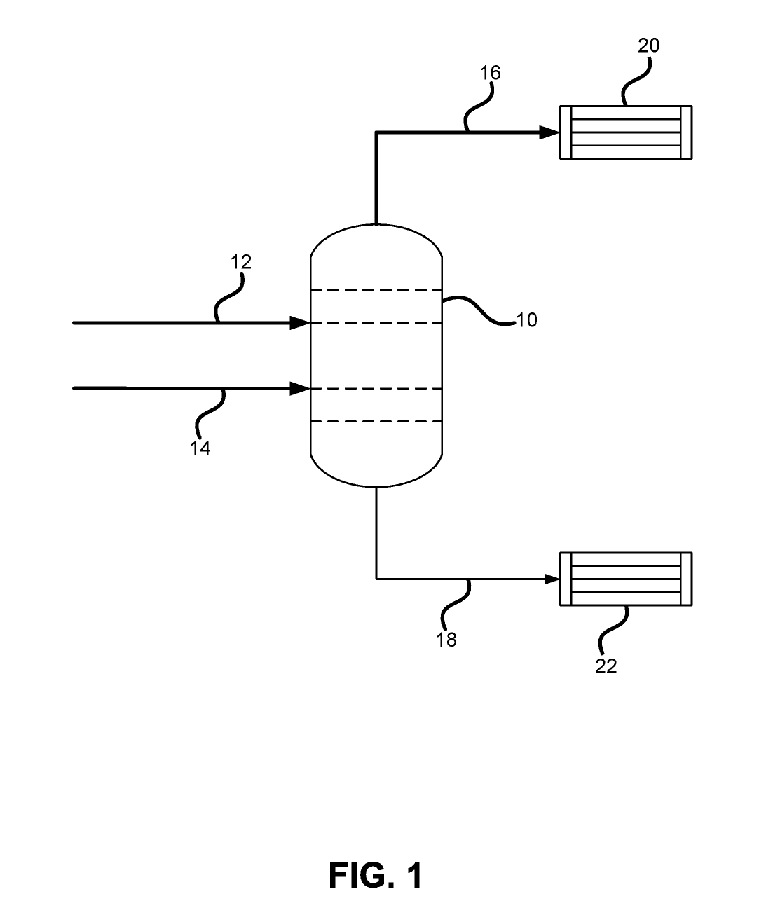

Referring to FIG. 1, a distillation column is provided, which may include a reboiler and/or other typical components. A first input feed in the form of a mixture of 1233zd(Z) and 244fa is provided to column 10 at 12. This mixture may include as little as 5.0 wt. %, 15.0 wt. %, or 30.0 wt. %, or as great as 80.0 wt. %, 90.0 wt. %, or 98.0 wt. % 1233zd(Z), and may include as little as 2.0 wt. %, 10.0 wt. %, or 20.0 wt. %, or as great as 70 wt. %, 85 wt. %, or 95 wt. % 244fa, based on the combined weight of 1233zd(Z) and 244fa, or may have amounts of 1233zd(Z) and 244fa within any range delimited by any pair of the foregoing values set forth in this paragraph, such as between 80.0 wt. % and 98.0 wt. % or between 90.0 wt. % and 98.0 wt. % for 1233zd(Z), for example, and between 2.0 wt. % and 20.0 wt. %, between 2.0 wt. % and 10.0 wt. %, for example, for 244fa.

Then, a second input feed of HF is provided to column 10 at 14. The amount of HF added may be as little as 1.0 wt. %, 1.75 wt. %, or 10.3 wt. %, or as great as 18.0 wt. %, 19.6 wt. %, or 34.3 wt. %, based on the combined weights of 1233zd(Z), 244fa, and HF, or the amount of HF that may be added may be within any range delimited by any pair of the foregoing values set forth in this paragraph, such as between 1.0 wt. % and 34.3 wt. %, between 1.75 wt. % and 19.6 wt. %, or between 10.3 wt. % and 18.0 wt. %, for example.

Upon addition of HF to column 10, a binary azeotrope of 1233zd(Z) and HF is formed. 1233zd(Z) has a boiling point of about 39.5° C. and HF has a boiling point of about 20° C. at standard atmospheric pressure. The thermodynamic state of a fluid is defined by its pressure, temperature, liquid composition and vapor composition. For a true azeotropic composition, the liquid composition and vapor phase are essentially equal at a given temperature and pressure range. In practical terms this means that the components cannot be separated during a phase change. For the purpose of this invention, an azeotrope is a liquid mixture that exhibits a maximum or minimum boiling point relative to the boiling points of surrounding mixture compositions. Also, as used herein, the term “azeotrope-like” refers to compositions that are strictly azeotropic and/or that generally behave like azeotropic mixtures.

An azeotrope or an azeotrope-like composition is an admixture of two or more different components which, when in liquid form under a given pressure, will boil at a substantially constant temperature, which temperature may be higher or lower than the boiling temperatures of the individual components and which will provide a vapor composition essentially identical to the liquid composition undergoing boiling.

For the purpose of this invention, azeotropic compositions are defined to include azeotrope-like compositions, which is a composition that behaves like an azeotrope, i.e., has constant boiling characteristics or a tendency not to fractionate upon boiling or evaporation. Thus, the composition of the vapor formed during boiling or evaporation is the same as or substantially the same as the original liquid composition. Hence, during boiling or evaporation, the liquid composition, if it changes at all, changes only to a minimal or negligible extent. This is in contrast with non-azeotrope-like compositions in which during boiling or evaporation, the liquid composition changes to a substantial degree.

Accordingly, the essential features of an azeotrope or an azeotrope-like composition are that at a given pressure, the boiling point of the liquid composition is fixed and that the composition of the vapor above the boiling composition is essentially that of the boiling liquid composition, i.e., essentially no fractionation of the components of the liquid composition takes place. Both the boiling point and the weight percentages of each component of the azeotropic composition may change when the azeotrope or azeotrope-like liquid composition is subjected to boiling at different pressures. Thus, an azeotrope or an azeotrope-like composition may be defined in terms of the relationship that exists between its components or in terms of the compositional ranges of the components or in terms of exact weight percentages of each component of the composition characterized by a fixed boiling point at a specified pressure.

The present invention provides a composition which comprises effective amounts of 1233zd(Z) and HF to form an azeotropic or azeotrope-like composition. As used herein, the term “effective amount” is an amount of each component which, when combined with the other component, results in the formation of an azeotrope or azeotrope-like mixture.

The binary azeotropes will typically consist essentially of combinations of 1233zd(Z) and HF, or consist of combinations of 1233zd(Z) and HF. As used herein, the term “consisting essentially of”, with respect to the components of an azeotrope-like composition or mixture, means the composition contains the indicated components in an azeotrope-like ratio, and may contain additional components provided that the additional components do not form new azeotrope-like systems. For example, azeotrope-like mixtures consisting essentially of two compounds are those that form binary azeotropes, which optionally may include one or more additional components, provided that the additional components do not render the mixture non-azeotropic and do not form an azeotrope with either or both of the compounds (e.g.; do not form a ternary azeotrope).

The 1233zd(Z)/HF azeotrope formed may include as little as 65.0 wt. %, 67.5 wt. %, or 70.0 wt. %, or as great as 75.0 wt. %, 77.5 wt. %, or 80.0 wt. % 1233zd(Z), and may include as little as 20.0 wt. %, 22.5 wt. %, or 25.0 wt. %, or as great as 30.5 wt. %, 32.5 wt. %, or 35.0 wt. % HF, based on the combined weight of 1233zd(Z) and HF, or may have amounts of 1233zd(Z) and HF within any range delimited by any pair of the foregoing values set forth in this paragraph, such as between 65.0 wt. % and 80.0 wt. %, between 67.5 wt. % and 77.5 wt. %, or between 70.0 wt. % and 75.0 wt. % for 1233zd(Z), and between 20.0 wt. % and 35.0 wt. %, between 22.5 wt. % and 32.5 wt. %, or between 25.0 wt. % and 30.5 wt. for HF, for example.

The azeotropic or azeotrope-like mixture of 1233zd(Z)) and HF has a boiling point of from about 0° C. to about 60° C. at a pressure of about 3 psia to about 73 psia when the hydrogen fluoride is present in an amount of from about 20 about 35 wt. %. For example, an azeotropic or azeotrope-like composition of 1233zd(Z) and HF having about 26±3 wt. % HF and about 74±3 wt. % 1233zd(Z) has been found to boil at about 25° C. and 14.7 psia.

The distillation may be conducted at pressure of as little as 1 psig, 3 psig, or 5 psig, or as great as 114.7 psig, 164.7 psig, or 214.7 psig, or may be conducted at a pressure within any range delimited by any pair of the foregoing values set forth in this paragraph, such as between 1 psig and 214.7 psig, between 3 psig and 164.7 psig, or between 5 psig and 114.7 psig.

After the 1233zd(Z)/HF azeotrope is formed in column 10, a distillate including mostly the 1233zd(Z)/HF azeotrope with minor amounts of 244fa may then be removed as an overhead stream 16 from the top of the distillation column. This distillate may include a reduced or minor amount 244fa, in particular, the distillate may include less than 5.0 wt. % 244fa, less than 1.0 wt. % 244fa, less than 0.5 wt. % 244fa, or less than 0.1 wt. % 244fa.

Also, after the 1233zd(Z)/HF azeotrope is formed in column 10, a bottoms stream 18 including mostly 244fa with minor amounts of 1233zd(Z) and HF may then be removed from the bottom of the distillation column. In a batch distillation, this bottoms stream may include a reduced or minor amount of 1233zd(Z), in particular, the bottoms stream may include less than 30.0 wt. % 1233zd(Z), less than 20.0 wt. % 1233zd(Z), less than 10.0 wt. % 1233zd(Z), less than 5.0 wt. % 1233zd(Z), or less than 1.0 wt. % 1233zd(Z). These amounts will depend on how much material is charged to the batch distillation column in view of the volume of the distillation column and how much distillate is removed (or how much material is left in the reboiler after some distillate is removed). The concentration of 1233zd(Z) in the reboiler will decrease continuously as it is removed in the overhead stream.

Additional HF may be added continuously to the distillation column 10 to make up for HF that is removed as distillate and ensure a relatively constant distillate composition. If the distillation is performed in batch mode, make-up HF feed is continued until a desired purity of 244fa is achieved in the column 10. If the distillation is performed in continuous mode, make-up HF and 1233zd(Z)/244fa mixture in desired proportions are fed continuously to maintain a desired purity of 244fa in the continuous distillation bottoms stream.

Other impurities that may exist in the 1233zd(Z)/244fa may be separated prior to input of the mixture into column 10 and/or may be separated within column 10 upon removal of the 1233zd(Z)/HF azeotrope as overhead stream 16. Such impurities may include 1,1,1,3,3-pentachloropropane (240fa), 1,1,3,3tetrachloro-1-fluoropropane (HFC-241fa), 1,3,3-trichloro-1,1-difluoropropane (242fa), 3,3-dichloro-1,1,1-trifluoropropane (243fa), 1,3-dichloro-3,3-difluoroprop-1-ene (1232zd), (E)-1-chloro-3,3,3trifluoropropene (1233zd(E)), (E)-1,3,3,3-tetrafluoropropene (1234ze(E)), (Z)-1,3,3,3-tetrafluoropropene (1234ze(Z)), and 1,1,1,3,3-pentafluoropropane (245fa), and the overhead stream 16 may include less than 5.0 wt. %, less than 1.0 wt. %, less than 0.5 wt. %, or less than 0.1 wt. % of a combined total of such impurities and/or a combined total of all compounds other than 1233zd(Z), HF, and 244fa.

In a further embodiment, shown at 20 in FIG. 1, following removal of the overhead stream 16, the 1233zd(Z) and HF components of the 1233zd(Z)/HF azeotropic composition may be separated to produce a purified form of 1233zd(Z) which is essentially HF-free. As used herein, “essentially HF-free” or “HF-free” refers to compositions of 1233zd(Z) which include less than 1.0 wt. % HF, less than 0.5 wt. % HF, or less than 0.1 wt. % HF.

Separation methods may include any method generally known in the art. In one embodiment, for example, the excess HF can be removed from the 1233zd(Z) by liquid-liquid phase separation, though other alternatives include distillation or scrubbing. The remaining HF can then be removed from the 1233zd(Z) by distillation and/or the use of one or more drying media or desiccants such as molecular sieves, calcium sulfate, silica, alumina, and combinations thereof. Purified 1233zd(Z) may be used as an end product such as a refrigerant, blowing agent, propellant, or diluent for gaseous sterilization, or it may be used as a starting material, an intermediate, a monomer, or otherwise further processed for the production of alternative HFOs or similar compounds.

In further embodiments, shown at 22 in FIG. 1, the bottoms stream of relatively pure 244fa can be subjected to a dehydrofluorination reaction for conversion to (E)-1-chloro-3,3,3-trifluoropropene (HCFO-1233zd(E)) or alternatively, the bottoms stream of relatively pure 244fa can be subjected to a fluorination reaction for conversion to 1,1,1,3,3-pentafluoropropane (HFC-245fa).

The following non-limiting Examples serve to illustrate the invention.

### Comparative Example 1

A distillation column (2-liter reboiler, 1 inch internal diameter Inconel column packed with ⅛ inch Inconel Helicoil packing material to a packed height 40 inches) was charged with about 900 g of 244fa/1233zd(Z) mixture with the ratio 244fa:1233zd(Z)=26:72. The distillation of the mixture was conducted at a pressure in the range of from about 7 psig to about 10 psig. The distillate collection rate was maintained in the range of from about 10 g/hr to about 15 g/hr. Under these conditions, a total 120 g of distillate were collected, and the 1233zd(Z) was only slightly concentrated in the overhead stream (distillate) relative to the remaining composition in the reboiler. Specifically, the ratio 244fa:1233zd(Z) in distillate was about 14:86 while in the reboiler the ratio 244fa:1233zd(Z) was about 28:71.

### Example 2

After the experiment of Comparative Example 1 was completed, 98 g of anhydrous hydrogen fluoride (HF) were added to the reboiler. The distillation column was then stabilized at a pressure of 20 psig. At these conditions, the distillate was collected at a rate of about 12 g/hr. After collecting about 40 g of the distillate, the HF was absorbed using distilled water and the organic portion of the distillate was analyzed using gas chromotography (GC), revealing that less than about 1 GC area % 244fa was present in the distillate, while the concentration of 1233zd(Z) in the distillate was 98.5 GC area %.

Thereafter, the distillation was continued and the distillate was analyzed after collecting an additional 40 g of distillate, which was determined via GC to have 0.46 GC area % 244fa and 99.1 GC area % 1233zd(Z). Also, at the same time as the foregoing sample was taken, a sample from the reboiler indicated that 244fa was concentrated in the reboiler, specifically, such sample included 34.6 GC area % 244fa and 65.3 GC area % 1233zd(Z). This experiment shows that 244fa can be effectively isolated from the 244fa/1233zd(Z) mixture by adding anhydrous hydrogen fluoride to the reboiler during distillation.

### Example 3

90 lbs. of a 1233zd(Z)/244fa mixture (75 wt. % 1233zd(Z), 25 wt. % 244fa) were charged to a distillation column (10 gallon reboiler, 2 inch internal diameter by 8 feet length propack column with a shell and tube condenser). The column had about 30 theoretical plates, and was equipped with temperature, pressure, and differential pressure transmitters. Then, 8 pounds of anhydrous hydrogen fluoride was added to the reboiler and the distillation column was stabilized at the pressure in the range from 20 to 25 psig. The distillate take-off rate was maintained in the range from 0.5 to 1.2 lb/hr. At these conditions the distillate contained only a small amount of 244fa (0.5-2.5 GC area %).

As the HF was depleted from the distillation column, an additional 20.1 lb. and then 8.5 lb. of anhydrous hydrogen fluoride was added to the reboiler. After the concentration of 244fa in the reboiler increased to about 80 GC area %, the distillation was stopped and total of 31 lbs. of material containing 20 GC area % 1233zd(Z) and 80 GC area % 244fa were collected from the reboiler.

### Example 4

The distillation column described in Example 2 was charged with 63.5 lbs. of a 1233zd(Z)/244fa/243fa mixture (68 wt. % 1233zd(Z)/30 wt. % 244fa/2 wt. % 243fa) and then 12 lb. of anhydrous hydrogen fluoride was added to the reboiler. The distillation column was run at a pressure in the range from 20 to 25 psig and the distillate take-off rate was maintained in the range from 0.5 to 1.2 lb/hr. As the HF was depleted, an additional 17 lb. of anhydrous hydrogen fluoride was added to the reboiler. At these conditions, the concentration of 244fa in the distillate was below 3 GC area % until the concentration of 1233zd(Z) in the reboiler decreased to below 15 GC area %.

### Example 5

After completion of the experiment of Example 4, 31 lbs. of material that was discharged from the distillation column at the end of the experiment of Example 3 was added to the reboiler. The resulting composition of the organic material in the reboiler was about 14.2 GC area % 1233zd(Z)/84.5 GC area % HCFC-244fa/0.8 GC area % HCFC-243fa. Then, an additional 5.3 lb. of HF was added to the distillation column reboiler. The distillation was performed at the pressure of about 20 psig and the distillate take-off rate was maintained in the range from 0.25 to 1.0 lb/hr. At these conditions, the concentration of 244fa in the distillate was below 3 GC area % until the the concentration of 1233zd(Z) in the reboiler decreased to below 5 GC area %.

After HF and 1233zd(Z) were distilled out of the column a total of 11 lb. of 99.5 wt. % pure 244fa and 17 lb. of 99.92 wt. % pure 244fa were collected in two portions. The composition of 99.9 wt. % pure 244fa was verified by ion chromatography (IC), gas chromatography (GC), and gas chromatography/mass spectroscopy (GC/MS) as follows: 99.9 wt. % 244fa, 250 ppm HFC-245fa, 80 ppm pentafluorobutane, 30 ppm pentafluorobutene, 80 ppm HCFO-1233zd(E), 40 ppm HCFC-235-isomer, 100 ppm HCFC-253 isomer, 200 ppm 1233zd(Z), and less than 80 ppm HF.

### Example 6

A low pressure distillation of a 244fa/1233zd(Z)/HF mixture was performed in a 1″ batch distillation column equipped with a 2 liter reboiler and condenser. The distillation column had an estimated 60 theoretical stages and was run at a pressure range of 12-15 psig. 1820 grams of organic (96.92/3.06 GC area % 1233zd(Z)/244fa) and 468 grams HF were charged to the distillation column, resulting in a 79.5/20.5 wt. % mixture of organic to HF. The starting amounts of 244fa and 1233zd(Z) in the distillation column were 3.06 GC area % and 96.92 GC area %, and the ending amounts of 244fa and 1233zd(Z) in the distillation column were 5.35 GC area % and 94.49 GC area %, respectively.

Distillation data corroborated the examples above as the 1233zd(Z) concentrated in the overhead and the bottoms became enriched in 244fa. The 244fa concentration in the overhead samples was <200 ppm in several samples, as shown in Table 1 below.

TABLE 1

| | Bottom of | | Top of distillation | | Overhead of | |
|---|---|---|---|---|---|---|
| | distillation column | | column | | distillation column | |
| | 244fa | 1233zd(Z) | 244fa | 1233zd(Z) | 244fa | 1233zd(Z) |
| | GC | GC | GC | GC | GC | GC |
| Time | area % | area % | area % | area % | area % | area % |
| 0400 | 3.56 | 96.44 | 0.04 | 99.96 | 0.1789 | 99.82 |
| 1200 | 3.94 | 96.06 | 0.17 | 99.83 | 0.0114 | 99.99 |
| 1830 | 4.24 | 95.76 | 0.04 | 99.96 | 0.0150 | 99.99 |
| 1100 | 3.77 | 96.23 | 0.07 | 99.93 | 0.0188 | 99.98 |
| 0630 | 4.31 | 95.69 | 0.15 | 99.85 | 0.0602 | 99.94 |
| 1700 | 4.01 | 95.99 | 0.02 | 99.98 | 0.0088 | 99.99 |
| 330 | 4.56 | 95.44 | 0.24 | 99.76 | 0.0661 | 99.93 |

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

It should be understood that the foregoing description is only illustrative of the present invention. Various alternatives and modifications can be devised by those skilled in the art without departing from the invention. Accordingly, the present invention is intended to embrace all such alternatives, modifications and variances that fall within the scope of the appended claims.

What is claimed is:

1. A method of separating (Z)-1-chloro-3,3,3-trifluoropropene (HCFO-1233zd(Z)) and 1-chloro-1,3,3,3-tetrafluoropropane (HCFC-244fa), comprising the steps of:
providing a mixture of 1233zd(Z) and 244fa to a distillation column;
adding an amount of hydrogen fluoride (HF) to the distillation column to form an azeotropic or azeotrope-like mixture consisting essentially of 1233zd(Z) and HF;
distilling the 244fa and the azeotrope-like mixture of 1233zd(Z) and HF; and
recovering the azeotropic or azeotrope-like mixture of 1233zd(Z) and HF in an overhead stream.

2. The method of claim 1, further comprising, after said distilling step, the additional step of recovering 244fa in a bottoms stream.

3. The method of claim 1, wherein said step of providing a mixture of 1233zd(Z) and 244fa further comprises providing a mixture of 1233zd(Z) and 244fa having between 5.0 wt. % and 98.0 wt. % 1233zd(Z) and between 2.0 wt. % and 95.0 wt. % 244fa, based on a combined weight of 1233zd(Z) and 244fa.

4. The method of claim 1, wherein said step of adding an amount of hydrogen fluoride (HF) further comprises adding between 1.0 wt. % and 34.3 wt. % HF, based on a combined weight of 1233zd(Z), 244fa, and HF.

5. The method of claim 1, wherein said distilling step is conducted at a pressure between 1 psig and 214.7 psig.

6. The method of claim 1, wherein the azeotropic or azeotrope-like mixture of 1233zd(Z) and HF has a boiling point of about 0-60° C. at a pressure of about 3-73 psia.

7. The method of claim 1, wherein said step of recovering the azeotropic or azeotrope-like mixture of 1233zd(Z) and HF in an overhead stream further comprises recovering less than 5.0 wt. % 244fa in the overhead stream.

8. The method of claim 1, wherein said step of recovering the azeotropic or azeotrope-like mixture of 1233zd(Z) and HF in an overhead stream further comprises recovering less than 1.0 wt. % 244fa in the overhead stream.

9. The method of claim 2, wherein said step of recovering 244fa in a bottoms stream further comprises recovering 244fa that includes less than 20.0 wt. % 1233zd(Z).

10. The method of claim 2, wherein said step of recovering 244fa in a bottoms stream further comprises recovering 244fa that includes less than 10.0 wt. % 1233zd(Z).

11. The method of claim 1, wherein said method is a continuous process and further comprises the additional steps of repeating said recovering steps and adding an additional amount of HF to make up for HF removed in said recovering steps.

12. The method of claim 1, further comprising, after said step of recovering the azeotropic or azeotrope-like mixture of 1233zd(Z) and HF in an overhead stream, the additional step of separating the 1233zd(Z) and HF.

13. The method of claim 1, further comprising, after said step of recovering 244fa, an additional step selected from the group consisting of:
subjecting the recovered 244fa to a dehydrofluorination reaction to convert the 244fa to (E)-1-chloro-3,3,3-trifluoropropene (HCFO-1233zd(E)); and
subjecting the recovered 244fa to a fluorination reaction to convert the 244fa to 1,1,1,3,3-pentafluoropropane (HFC-245fa).

14. A method of separating (Z)-1-chloro-3,3,3-trifluoropropene (HCFO-1233zd(Z)) and 1-chloro-1,3,3,3-tetrafluoropropane (HCFC-244fa), comprising the steps of:
providing a mixture of 1233zd(Z) and 244fa to a distillation column, the mixture including between 5.0 wt. % and 98.0 wt. % 1233zd(Z) and between 2.0 wt. % and 95.0 wt. % 244fa, based on a combined weight of 1233zd(Z) and 244fa;
adding between 1.0 wt. % and 34.3 wt. % hydrogen fluoride (HF), based on a combined weight of 1233zd(Z), 244fa, and HF, to the distillation column to form an azeotropic or azeotrope-like mixture consisting essentially of 1233zd(Z) and HF having a boiling point of about 0-60° C. at a pressure of about 3-73 psia;
distilling the 244fa and the azeotrope-like mixture of 1233zd(Z) and HF;
recovering the azeotropic or azeotrope-like mixture of 1233zd(Z) and HF in an overhead stream; and
recovering 244fa in a bottoms stream.

15. The method of claim 14, wherein said step of recovering the azeotropic or azeotrope-like mixture of 1233zd(Z) and HF in an overhead stream further comprises recovering less than 5.0 wt. % 244fa in the overhead stream.

16. The method of claim 14, wherein said step of recovering the azeotropic or azeotrope-like mixture of 1233zd(Z) and HF in an overhead stream further comprises recovering less than 1.0 wt. % 244fa in the overhead stream.

17. The method of claim 14, wherein said step of recovering 244fa in a bottoms stream comprises recovering 244fa that includes less than 20.0 wt. % 1233zd(Z).

18. The method of claim 14, wherein said step of recovering 244fa in a bottoms stream comprises recovering 244fa that includes less than 10.0 wt. % 1233zd(Z).

19. The method of claim 14, further comprising, after said step of recovering the azeotropic or azeotrope-like mixture of 1233zd(Z) and HF in an overhead stream, the additional step of separating the 1233zd(Z) and HF.

20. The method of claim 14, further comprising, after said step of recovering 244fa, an additional step selected from the group consisting of:
subjecting the recovered 244fa to a dehydrofluorination reaction to convert the 244fa to (E)-1-chloro-3,3,3-trifluoropropene (HCFO-1233zd(E)); and
subjecting the recovered 244fa to a fluorination reaction to convert the 244fa to 1,1,1,3,3-pentafluoropropane (HFC-245fa).
